(19) United States
(12) Patent Application Publication
(10) Pub. No.: US 2017/0231916 A1
(43) Pub. Date: Aug. 17, 2017

(54) COMPACTED SOLID DOSAGE FORM

(71) Applicant: Janssen Sciences Ireland UC, Little Island, Co Cork (IE)

(72) Inventors: Henderik Willem FRIJLINK, Eelde (NL); Niel GRASMEIJER, Assen (NL); Wouter Leonardus Joseph HINRICHS, Groningen (NL); Katie Ingrid Eduard AMSSOMS, Hove (BE); Lieven Elvire Colette BAERT, Brugge (BE)

(73) Assignee: Janssen Sciences Ireland UC

(21) Appl. No.: 15/501,425

(22) PCT Filed: Aug. 3, 2015
(86) PCT No.: PCT/EP2015/067770

§ 371 Date: Feb. 2, 2017

(30) Foreign Application Priority Data
Aug. 4, 2014 (EP) 14179691.2

(51) Int. Cl. A61K 9/20; A61K 31/721; A61K 31/522

(52) U.S. Cl. CPC A61K 9/204; A61K 31/522; A61K 31/721; A61K 9/2018; A61K 9/2027

(57) ABSTRACT

The present invention relates to dosage forms comprising a compressed blend of a biologically active ingredient, one or more polymers like a poly(a-hydroxy carboxylic acid) in which optionally is incorporated a glass transition modifying agent, and optional further ingredients, wherein the polymer or polymeric mixture has a specific glass transition temperature which causes the system to be in the glassy state at ambient conditions before administration and to be in the rubbery state under the physiological conditions to which the system is exposed after administration, resulting in pulsed release of said biologically active ingredient.

The present invention concerns dosage forms comprising a compressed blend of one or more biologically active ingredients, one or more polymers like a poly(a-hydroxy carboxylic acid) in which optionally is incorporated a glass transition modifying agent, and optional further ingredients, wherein the polymer or polymeric mixture has a specific glass transition temperature which causes the system to be in the glassy state at ambient conditions before administration and to be in the rubbery state under physiological conditions to which the system is exposed after administration, resulting in pulsed release of said biologically active ingredient(s).

In order to meet specific therapeutic requirements many drugs are formulated in controlled release dosage forms. These include variants such as sustained release where prolonged release is intended, delayed release where drug release starts after a predetermined period of time and pulsed release. Sustained release aims to maintain a nearly constant drug concentration in the therapeutic window for prolonged time by a slow and steady release into the bloodstream. In pulsed (or pulsatile) drug delivery a specific lag time during which little or no drug is released is followed by the transient release of the active ingredient within a short period of time.

Pulsed release can be induced by various mechanisms. In triggered delivery systems, the release is governed by changes in the physiological environment of the device (biologically triggered systems) or by external stimuli (such as the application of ultrasound, laser light, electrical impulses, pH or temperature changes, application of magnetic fields). In programmed delivery systems the release is completely governed by an inner mechanism of the device, i.e., the lag time prior to drug release is controlled primarily by the delivery system. Polymer based systems have been developed to this purpose including those that use a barrier technology that is placed around the active agent that is designed to degrade or dissolve after a certain time interval, and those that use the degradation of the polymer itself to induce the release of the active agent. Examples of the former are dosage forms having a drug-containing core with a polymer coating and of the latter dosage forms of a drug embedded in a bulk-eroding polymeric matrix.

There are many drugs that are more effective when given to the patient in a pulsatile manner as opposed to a continuous release fashion. Conditions to be treated may follow certain cyclic rhythms and the timing of medication regimens can improve the outcome of the therapy. Pharmacokinetics, drug efficacy and side effects can be modified by adjusting therapy to the biological rhythm of the patient. An optimal drug effect can thus be achieved by administering the drug at particular points in time. Pulsatile release can help in increasing patient compliance by reducing the number of administrations and simplifying the dosing scheme.

An area where pulsatile delivery is applied is that of vaccines. Many vaccines require an initial immunization followed by one or more booster immunizations at specific time intervals to assure complete protection. Often vaccination is ineffective by failure to obey the required time intervals or by missing booster immunizations. Although in developed countries programs have been set up to reassure that vaccination schemes are followed, there still are several instances for failure to receive complete immunization. These include poor, remote or limited access to medical care, lack of patient awareness and cultural or societal misconceptions about vaccines and vaccination as such. Particularly in developing countries these problems are exacerbated so that patients do not receive the required booster immunizations. It would be more economical and effective, especially in third world countries, if a vaccine could be implanted once into the patient and the boosters be released automatic and/or pre-programmed from the implanted or injected device.

A single-administration vaccine (SAV) may provide an adequate solution to these problems. Several approaches for SAVs have been proposed amongst which controlled release vaccines that release organism antigens at selected times has shown the most promising for achieving a SAV. In the latter approach the repeated administrations are provided automatically. Several different approaches have been developed to this purpose such as liposomes, unilamellar vesicles, emulsions and polymers. As a result of the instability of liposomes, lipid vesicles and emulsions in vivo, these systems yield only an initial exposure to the antigen, and a booster immunization is usually required to achieve protection against disease.

Polymer base systems have been developed that are time-controlled including those that use a barrier technology that is placed around the active agent that is designed to degrade or dissolve after a certain time interval, and those that use the degradation of the polymer itself to induce the release of the active agent.

Injectable biodegradable polymer formulations for vaccine delivery were found an attractive option for development as SAVs (reviewed by Cleland, Trends in Biotechnology, Vol. 17, pp 25-29 (1999)). Several polymer types have been investigated but systems in which the vaccine is incorporated in poly(lactic-co-glycolic acid) (PLGA) microspheres were considered a promising approach. Upon contact of the dried microspheres with bodily fluids, the antigen diffuses out of the surface portion of the microspheres into the surrounding environment. This initial release of antigen may then be followed by either continued diffusion of the antigen out of the microspheres (continuous release) or a lag phase caused by lack of pores or channels for antigen diffusion (pulsatile release). Water-catalyzed ester hydrolysis of the PLGA results in a collapse of the polymer matrix resulting in a second pulse due to bulk release. The time of this pulse is dependent upon the rate of polymer degradation, which is dictated by the polymer's composition and molecular weight.

In order to trap the vaccine in polymer both the vaccine and polymer need to be mixed. Aqueous solutions cannot be used because the PLGA polymer is not water-soluble while co-melting or the use of organic solvents affects the integrity of the vaccine. Another problem associated with this type of systems is to obtain sufficiently high vaccine loading. Also the stability and structural integrity of the vaccine embedded within the polymeric matrix is problematic.

Although this controlled-release technology held promises to provide complete protection against a disease after a single administration, it never could sufficiently mimic the separate administration scheme of traditional vaccinations, thereby failing to provide complete immunization.

There is a need for alternative approaches for existing pulsed delivery of biologically active ingredients. In particular there is a need for compositions that upon an initial release of the biologically active ingredient, release the biologically active ingredient in a next pulse preferably after a certain period of time during which time little or no active ingredient is released.

There is a particular need for single-administration vaccines that provide complete and long-lasting protection following a single immunization administration.

It now has been found that a dosage form made of a compacted composition comprising a mixture of alone or more biologically active ingredients, one or more polymers having a glass transition temperature which causes the system to be in the glassy state at ambient conditions before administration and to be in the rubbery state under physiological conditions to which the system is exposed after administration, and optional further ingredients, a two pulse release is obtained.

This invention concerns a compacted solid dosage form comprising a compressed blend of one or more biologically active ingredients, one or more biocompatible, biodegradable polymers like poly(a-hydroxy carboxylic acid) in which, optionally, is incorporated a glass transition modifying agent (plasticizer or anti-plasticizer), and optional further ingredients, wherein the polymer or polymeric mixture has such a glass transition temperature (Tg) that the material will be in the glassy state when it is kept at ambient conditions (or storage conditions).

In general this means that the Tg of the material will be over 30° C. when it is in the dry state and formulated with the drug and or other excipients. Next to this requirement, the Tg of the polymer or polymeric mixture should be low enough to assure that after administration (that is under physiological conditions) the material will be in the rubbery state. In general this means that the Tg of the material when immersed in an aqueous liquid will be below 38 to 40° C.

These requirements will, for example, thus be met by a polymer (or polymeric mixture) which in the dry state has a Tg of 52° C. and of which the Tg is lowered to 33° C. when the material is immersed in an aqueous solution. Another, example of a polymer or polymeric mixture that would meet these requirements is a polymer or polymeric mixture which during storage (dry) has a Tg of 35° C. and has a Tg of 35° C. under physiological conditions.

The glass transition modifying agent can be a plasticizer or an anti-plasticizer. Preferably, the poly a-hydroxy carboxylic acid is D,L-polylactic acid (PLA).

The compressed powdery mixture further contains a water-soluble filler, in particular a polyol such as mannitol.

The compressed powdery mixture may further contain a fructan, which in particular may be inulin.

The biologically active ingredient can be various but in one embodiment it is a vaccine. In a particular, the vaccine may be incorporated in an inulin matrix.

The present invention also concerns a method for the pulsatile or multistep pulsatile delivery of a biologically active ingredient to a patient in need thereof comprising administering to the patient a dosage form of the present invention comprising an effective amount of the biologically active ingredient.

The dosage forms of this invention can be advantageously prepared using simple methodology wherein the components are blended together and subsequently compressed into a dosage form of desired shape and size.

The selection of the polymer and the quantity of the ingredients of the dosage forms of the invention allows programming the timing and quantity of the release of the biologically active compounds at desired intervals. No or negligibly small quantities of the biologically active ingredient are released between the initial release and the second release pulse.

In case the biologically active ingredient is a vaccine, the dosage forms of this invention can be used as single-administration vaccines. They may provide complete and lasting immunization without the need of booster administrations. As such, the present invention also provides a method of immunizing a patient against a disease comprising administering to the patient a dosage form of the present invention containing an effective amount of a vaccine.

As used herein, the terms “active ingredient” and “biologically active ingredient” are meant to have the same meaning and are used interchangeably. The term “active ingredient” refers to any (biologically) active ingredient, including pharmaceutical active ingredients, vaccins, neutraceuticals, and cosmeceuticals. The terms “vaccins” refers to specific antigens, subunits, nucleic acids or any other material that elicits an immune response against viruses, fungi, bacteria, and other infectious or non-infectious pathogens. The terms “pharmaceutical active ingredient” and “drug” are meant to be equivalent. Drugs can be for human or for veterinary use. Pharmaceutical active ingredients comprise synthetic molecules, biomolecules, antibodies, and the like. Neutraceuticals are active ingredients used in nutrition and include ingredients that have an effect on the general well-being.

These encompass food supplements such as, for example, dietary food supplements, vitamins, minerals, fiber, fatty acids, and amino acids. Examples of such ingredients are Vitamin C, omega-3 fatty acids, carotenes, and flavonoids. Cosmeceuticals include active ingredients that have an effect on the outer appearance of an individual such as on skin, hair, lips, and eyes, and encompass anti-wrinkling agents and agents that improve complexion.

The dosage forms of the invention may be referred to as a “compact” for administration of an active ingredient to a human or warm-blooded animal. The compacts may be for administration rectally, vaginally, or by implantation. They may take a variety of shapes and sizes, such as round, oblong, capsule-shaped, cylinder-shaped or other shapes.

If desired, the compact may be covered with a coating.

The poly(α-hydroxy carboxylic acid) for use in the invention is biocompatible and biodegradable meaning that it is non-toxic and the products resulting from its biodegradation are non-toxic as well and are readily eliminated from the body.

The poly(a-hydroxy carboxylic acid) can be an acid-terminated polyester of glycolic acid or of lactic acid, or a copolymer thereof such as polylactic acid (polylactide), polyglycolic acid (polyglycolide) or poly(lactic-co-glycolic acid). The lactic acid in these polymers or copolymers preferably is racemic, e.g. poly(D,L-lactic acid) (PDLLA), also referred to as poly(D,L-lactide) or simply by polylactic acid (PLA); or poly(D,L-lactic-co-glycolic acid) (PLGA), which is a copolymer of glycolic acid and of racemic lactic acid. Of interest for use in the invention is poly(D,L-lactic acid), also referred to as polylactide (PLA). The lactic acid in the poly(α-hydroxy carboxylic acid) polymers or copolymers may be chiral, e.g. poly(D-lactic acid) (PDLA) or poly(L- lactic acid) (PLLA), or a physical mixture thereof, or a copolymer of PDLA and PLLA. Also included is poly(L-lactic acid-co-D,L-lactic acid) (PLDLLA).

The polylactide for use in the dosage forms of the invention may have an intrinsic viscosity midpoint in chloroform that is in the range from 0.1-2 dl/g, or from 0.1 to 1 dl/g in particular from 0.16 to 0.24 dl/g. A suitable polymeric material for use in the dosage forms of the invention is an acid terminated poly-DL-lactide with an intrinsic viscosity midpoint of about 0.20 dl/g. An example of this material is available from the Purac division of CSM N.V. under the trademark PURASORB PDL-02. Intrinsic viscosity can be measured for these polymers using a 1.0 g/dl solution of the polymer in CHCl₃ in a capillary viscometer at 25° C.

If the poly(α-hydroxy carboxylic acid) is a copolymer of lactic and glycolic acid, said polymer may have an intrinsic iscosity of from 0.1 to 1 dl/g, in particular of from 0.14 to 0.22 dl/g.

If the poly(a-hydroxy carboxylic acid) is polyglycolide, said polymer may have an intrinsic viscosity of from 0.1 to 2 dl/g, in particular of from 1.0 to 1.6 dl/g.

The polylactic acid material may have a molecular weight of at least about 10 kD, preferably at least about 12 kD, or 15 kD, especially not more than about 150 kD, preferably not more than about 140 kD, especially not more than about 25 kD. Molecular weight values referred to herein are weight average molecular weights.

The polymer or polymeric mixture comprising a poly(α-hydroxy carboxylic acid) has a glass transition temperature (Tg) after implantation and/or administration which is in the range of about 30° C.—about 45° C., or in particular of about 30° C.—about 40° C., or of about 36° C.—about 38° C.

The poly(α-hydroxy carboxylic acid) is present in the dosage forms of the invention in an amount that is in the range from about 40% to 99.99%. The polymer or polymeric mixture comprising a poly(α-hydroxy carboxylic acid) does not contain active ingredient and vice versa the active ingredient does not contain polymer or polymeric mixture comprising a poly(α-hydroxy carboxylic acid), both unless in negligible quantities.

The polymer or polymeric mixture comprising a poly(α-hydroxy carboxylic acid) may contain a poly(α-hydroxy carboxylic acid) optionally mixed with a glass transition modifying agent and, optionally, one or more further ingredients. The glass transition modifying agent may be a plasticizer or an anti-plasticizer.

In case the poly(a-hydroxy carboxylic acid) has a Tg higher than the ranges specified above, one or more plasticizers may be added to lower the Tg until within the desired temperature range. In case the poly(α-hydroxy carboxylic acid) has a Tg lower than the ranges specified above, one or more anti-plasticizers may be added to increase the Tg until within the desired temperature range. The quantity of the plasticizer or anti-plasticizer to be added depends on the initial Tg of the poly(α-hydroxy carboxylic acid) and on the desired Tg of the polymer mixture. Said quantity may be in the range from 0% to 20 w/w %. Plasticizers that can be added include tributyl citrate, polyethylene glycol (PEG), glycerol, or castor oil. Of interest are biodegradable plasticizers. Preferred are tributyl citrate and PEG. Anti-plasticizers that can be added include triacetin, which is known to increase the Tg of poly(α-hydroxy carboxylic acid), is non-toxic, and safe for clinical use.

The compositions may in addition contain additives necessary to stabilize the active ingredient such as mannitol, a fructan such as inulin, or trehalose. These additives may be present in the dosage forms of the invention in an amount that is in the range from 0% to 60%. These additives in particular are fructans. As used herein, a fructan is understood to mean any oligo- or polysaccharide that contains a plurality of anhydrofructan units. The fructans can have a polydisperse chain length distribution and can have a straight or branched chain. Branched fructans are often designated as glucans. In the context of the present invention, these substances are also understood to fall within the term fructans.

Preferably, the fructans contain mainly β-1,2 bonds, as in inulin, but they can also contain β-2,6 bonds, as in levan. Suitable fructans can originate directly from a natural source, but may also have undergone a modification. Examples of modifications in this connection are reactions known per se that leading to a lengthening or shortening of the chain length.

An important parameter of fructans suitable according to the invention is the average chain length (number-average degree of polymerization, DPn). It should be at least 6 and will normally not be greater than about 1,000. Preferably, a fructan is used having a DPn of at least 7, more preferably at least 10, still more preferably at least 14, up to about 60. The inulin in the compositions of the invention has a degree of polymerization (DP) that is in the range of about 6 to about 60, in particular from about 10 to about 60. The DPn can be determined by methodology known in the art such as by High Pressure liquid Chromatography (anion exchange HPLC).

Fructans that are suitable according to the invention are, in addition to naturally occurring polysaccharides, also industrially prepared polysaccharides, such as hydrolysis products, which have shortened chains, and fractionated products having a modified chain length, in particular having a DPn of at least 6. A hydrolysis reaction to obtain a fructan having a shorter chain length can be carried out enzymatically (for instance with endoinulinase), chemically (for instance with aqueous acid), physically (for instance thermally) or by the use of heterogeneous catalysis (for instance with an acid ion exchanger). Fractionation of fructans, such as inulin, can be achieved inter alia through crystallization at low temperature, separation with column chromatography, membrane filtration, and selective precipitation with an alcohol. Other fructans, such as long-chain fructans, can be obtained, for instance through crystallization, from fructans from which mono- and disaccharides have been removed, and fructans whose chain length has been enzymatically extended can also serve as fructan that is used in the present invention. Further, reduced fructans can be used. These are fructans whose reducing end groups, normally fructose groups, have been reduced, for instance with sodium borohydride or hydrogen in the presence of a transition metal catalyst. Chemically modified fructans, such as crosslinked fructans and hydroxyalkylated fructans, can also be used.

The fructan for use in the invention may be inulin. Inulin is a polysaccharide, consisting of β-1,2 bound fructose units with an α-D-glucopyranose unit at the reducing end of the molecule. The substance occurs inter alia in the roots and tubers of plants of the Liliaceae and Compositae families. The most important sources for the production of inulin are the Jerusalem artichoke, the dahlia and the chicory root. Industrial production of inulin starts mainly from the chicory root. The main difference between inulin originating from the different natural sources resides in the degree of polymerization, which can vary from about 6 in Jerusalem artichokes to 10-14 in chicory roots and higher than 20 in dahlias.

Inulin is an oligosaccharide which in amorphous condition has favorable physicochemical properties for the application as auxiliary substance for pharmaceutical forms of administration. These physicochemical properties are: (adjustable) high glass transition temperature, low hygroscopicity, no (reducing) aldehyde groups and probably a low rate of crystallization. In addition, inulin is not toxic and readily available.

When a solution of inulin is dried, for instance by freeze-drying, vacuum-drying or spray-drying, amorphous inulin can be obtained. It has been found that if further a pharmacon is present in the solution, it is protected by inulin from harmful influences during drying, and that after the drying process the pharmacon is surrounded by a protective coating of amorphous inulin. As a result, it will be possible to considerably lengthen the shelf-life of unstable pharmacons, such as therapeutic proteins and peptides. In addition, with such a coating the bioavailability of poorly soluble pharmacons could be raised considerably.

In one embodiment, the active ingredient is a vaccine that is incorporated in inulin.

The dosage forms may in addition contain one or more water-soluble fillers, which may include polyols such as mannitol, sorbitol or sugars such as lactose, sucrose, glucose. The total amount of the fillers that is present in the dosage forms of the invention may be maximum 60%.

The biologically active ingredient may be small or large molecular, either synthetic, semi-synthetic or natural. Included are antibiotics, antimycotics, hormones, peptides, and proteins.

The biologically active ingredient in particular is a vaccine. Vaccines that can be incorporated in the dosage forms of the present invention include killed, but previously virulent, micro-organisms that have been destroyed with chemicals, heat, radioactivity, or antibiotics. Examples include influenza, cholera, bubonic plague, polio, hepatitis A, and rabies.

They further include attenuated microorganisms in particular attenuated viruses such as, for example, the viral diseases yellow fever, measles, rubella, and mumps, and the bacterial disease typhoid. Further included is the Mycobacterium tuberculosis vaccine. A further type includes the toxoid-based vaccines such as tetanus and diphtheria vaccines. Still a further class of vaccines are those based on protein subunits (or protein fragments). Examples include the subunit vaccine against Hepatitis B virus that is composed of only the surface proteins of the virus, the virus-like particle (VLP) vaccine against human papillomavirus (HPV) that is composed of the viral major capsid protein, and the hemagglutinin and neuraminidase subunits of the influenza virus, the subunit vaccine used for plague immunization. As a further type of vaccines there can be mentioned conjugates obtained by linking certain bacterial polysaccharide outer coats that are poorly immunogenic to proteins (e.g., toxins). This approach is used in the Haemophilus influenzae type B vaccine.

Of particular interest are vaccines against hepatitis B, rabies and pneumococcus.

In the instance of vaccines, the compositions of the present invention may contain further ingredients such as aluminium salts, in particular aluminium hydroxide or phosphate. In a particular embodiment, there is provided a solid dosage form comprising a compressed blend of a vaccine that is incorporated in inulin, poly(D,L-lactic acid) having a glass transition temperature that is in the range of about 30° C.—about 45° C., mannitol, and optional further ingredients. This solid dosage form may contain from 0% to 60% of said vaccine that is incorporated in inulin, from 40% to 99% of said poly(D,L-lactic acid), from 0% to 60% of mannitol, and optional ingredients up to 100%. The dosage forms of the invention are prepared by compaction of a blend of the various ingredients. The compression pressure in this compacting step may vary but in general is in the range from 1.6*10⁷ Pa to 2.3*10⁸ Pa, in particular from 7.9*10⁷ Pa to 2.0*10⁸ Pa.

The dosage forms of the invention can be prepared by mixing the ingredients followed by a compaction step to prepare the dosage form.

The ingredients in the blend prior to compression are present in particulate form. The average particle size of the ingredients will be small enough to ensure adequate compacting. If coarser materials are used they may be brought to the desired particle size by grinding. In particular when using an active ingredient incorporated into a fructan, in particular a vaccine incorporated into inulin, this ingredient is milled to the appropriate size. The solid dosage forms of the invention result in pulsed release. A first pulse of drug release occurs immediately after administration followed by a lag time after which a second pulse takes place. The time period between the initial release and the second pulse may vary and may be several days, such as 1-7 days; several weeks such as 1-6 weeks, in particular 1 to 3 weeks; or several months such as 1 to 6 months, in particular 1 to 3 months. The dosage forms of the present invention may also find application in the delivery of active ingredients to treat conditions or induce physiological changes in the body of the subject to which the dosage forms are administered, which conditions or physiological changes are susceptible to cyclic patterns. Examples include hormonal based drug delivery, fertility and birth control drug therapy for both animals and humans, which are not continuous, but rather cyclic in nature since these therapies work in conjunction with the menstrual cycle and the corresponding hormonal flux.

The dosage forms of the present invention may also find application in the vaginal delivery of various active ingredients such as hormones, anti-conceptives, anti-infectives, which may include anti-bacterials or antimycotics such as ketoconazole, fluconazole, itraconazole. In that instance the dosage forms will be shaped for local delivery such as, for example, as a vaginal ring.

The dosage forms of the present invention may also be used as coating on stents. The resulting drug-eluting stents show an initial release of the active ingredient immediately after positioning of the stent, a second peak is released after a predetermined lag period such as after 2-6 weeks. Active ingredients for this application include antiplatelet agents (anti-aggregants), antibiotics, anti-restenosis agents and anti-HCV compounds (especially for liver stents).

The compacted dosage forms can take various forms such as cylinders, tablets, concave or convex tablets, oblong tablets, rods or beads. Their size may vary but usually their largest dimension is in the millimeter range, for example 1 to 15 mm, although in case of rods their largest dimension may be in the centimeter range

The compacted dosage forms can be administered as implants such as by subcutaneous or intramuscular administration using a needle-like applicator such as a syringe or a trocar.

### EXAMPLE 1

A compact containing theophylline (model drug), mannitol, inulin and the polymer PLA (PDL-02), resulted in a biphasic release profile whereby a certain amount of theophylline was released immediately and a second amount of theophylline was released after a certain lag-time. The lag-time between the first and second release of theophylline and the released amount in each step depends on parameters like mannitol/inulin concentration and polymer type.

This biphasic release profile is obtained by physical mixing of the components and does not require a coating step.

### EXAMPLE 2

A compact containing dextran (model drug), mannitol, polyvinylpyrrolidone and the polymer PLGA (PDLG-5002), resulted in a biphasic release profile whereby a certain amount of dextran was released immediately and a second amount of dextran was released after a certain lag-time. The lag-time between the first and second release of dextran and the released amount in each step does not depend on the molar mass of the model drug.

This biphasic release profile is obtained by physical mixing of the components and does not require a coating step.

### DESCRIPTION OF THE DRAWINGS

FIG. 1 shows a compact wherein the release of theophylline from non-heated mixed implants compressed at 7.9*10⁷ and 2.0*10⁸ Pa. All implants consisted of PLA (IV 0.2) and contained mannitol.

In more detail it is an example of a compact wherein the release of theophylline from mixed implants compressed at 7.9*10⁷ (O) and 2.0*10⁸ (Δ) Pa is shown. All implants consisted of 90.9% PLA (IV 0.2), 5.1% mannitol, 3.6% inulin, and 0.4% theophylline. The inulin and theophylline was a freeze-dried powder mixture. Compressed implants were oblong 6×2×2 mm and were submerged in 37° C., 100 mM PBS release medium in a shaking water bath.

FIG. 2 shows a compact wherein the release of theophylline from implants with freeze dried inulin/theophylline, mixed with mannitol and implants with physically mixed inulin/theophylline, without mannitol compressed at 2.0*10⁸ Pa. All implants consisted of PLA (IV 0.2).

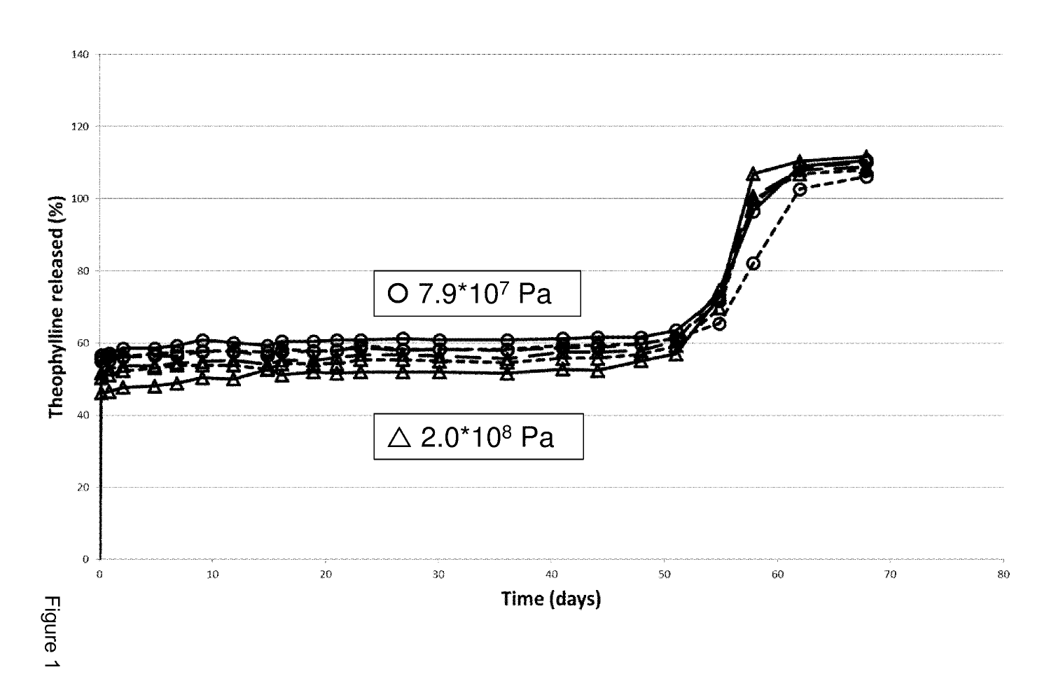

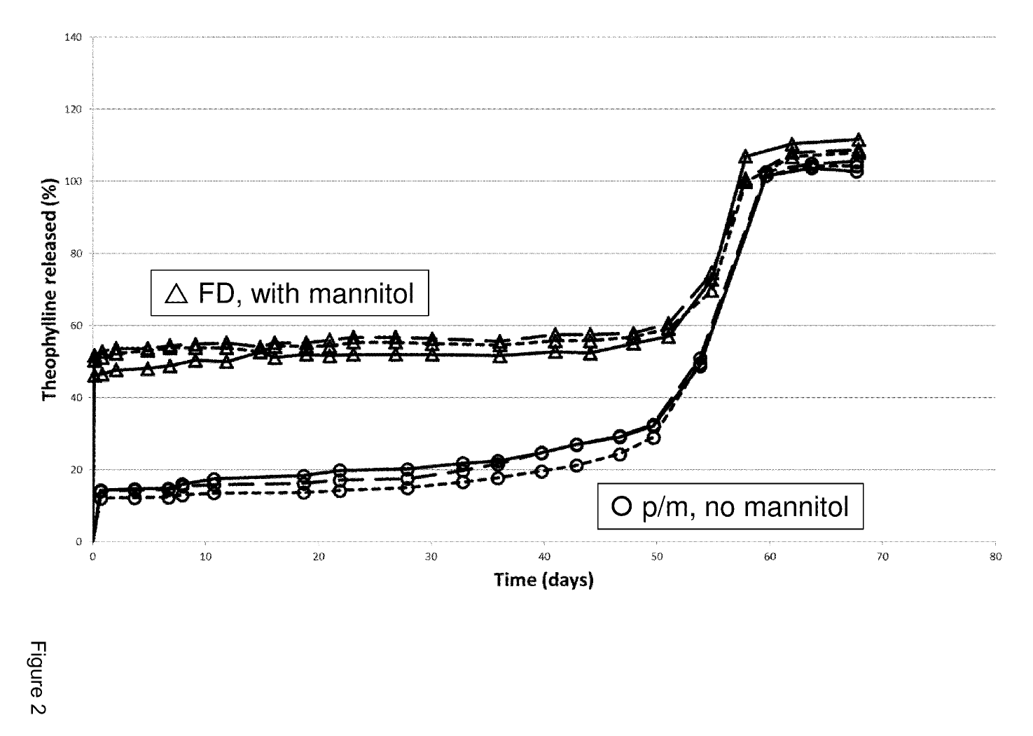

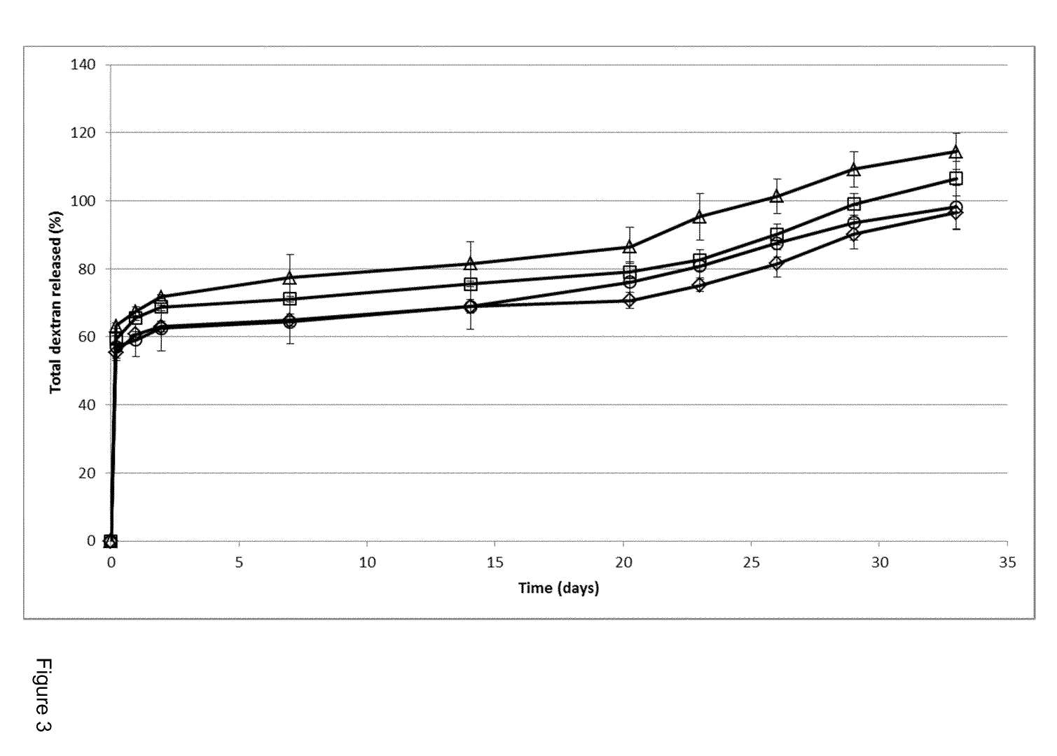

In more detail it is an example of a compact wherein the release of theophylline from implants with freeze dried inulin/theophylline, mixed with mannitol (Δ) and implants with physically mixed inulin/theophylline, without mannitol (◯) compressed at 2.0*10⁸ Pa is shown.

All implants contained 90.9% PLA (IV 0.2). For one implant the polymer was mixed with 5.1% mannitol, 3.6% inulin, and 0.4% theophylline, where the inulin and theophylline was a freeze-dried powder mixture. For a second implant the polymer was mixed with 8.3% inulin and 0.8% theophylline, where the inulin and theophylline was a physically mixed powder. Compressed implants were oblong 6×2×2 mm and were submerged in 37° C., 100 mM PBS release medium in a shaking water bath.

FIG. 3 shows a compact wherein the release of dextran with an average molar mass of 1000, 12000, 150000, and 1100000 Da from non-heated mixed implants with freeze dried polyvinylpyrrolidone (K12)/dextran, mixed with mannitol compressed at 2.0*10⁸ Pa. All implants consisted of PLGA (50:50 lactic:glycolic acid, IV 0.2).

More detailed it is an example of a compact wherein the release of dextran from implants with freeze dried polyvinylpyrrolidone/dextran (average molar mass 1000 (◯), 12000 (Δ), 150000 (□), and 1100000 (⋄) Da), mixed with mannitol compressed at 2.0*10⁸ Pa is shown. All implants contained 90.9% PLGA (50:50 lactic:glycolic acid, IV 0.2). For all implants the polymer was mixed with 5.1% mannitol, 3.6%polyvinylpyrrolidone, and 0.4% dextran, where the dextran and polyvinylpyrrolidone was a freeze-dried powder mixture. Compressed implants were oblong 6×2×2 mm and were submerged in 37° C., 100 mM PBS release medium in a shaking water bath.

1. A compacted composition comprising one or more biologically active ingredients and one or more polymers wherein the polymer or polymeric mixture has a specific glass transition temperature at ambient conditions before administration and at physiological conditions after administration, resulting in pulsed release of said one or more biologically active ingredient(s).

2. A composition according to claim 1 further comprising a glass transition modifying agent.

3. A composition according to claim 2 wherein the glass transition modifying agent is a plasticizer or an anti-plasticizer.

4. A composition according to claim 1 wherein the composition is a compacted solid composition.

5. A composition according to claim 4 wherein the one or more polymers is poly(α-hydroxy carboxylic acid).

6. A composition according to claim 1 further comprising a water-soluble filler.

7. A composition according to claim 1 wherein the one or more biologically active ingredients is a vaccine or vaccine component.

8. A method of immunizing a patient against a disease comprising administering to said patient a therapeutically effective composition according to claim 1.

9. A composition according to claim 1 for the use as a medicine or for the use as a means for delivering a medicament to a patient.
